(19) United States
(12) Patent Application Publication
(10) Pub. No.: US 2002/0131959 A1
(43) Pub. Date: Sep. 19, 2002

(54) Means and methods for the modulation of arteriogenesis

(76) Inventors: Ivo Buschmann, Freiburg (DE); Niels Van Royen, Gundelfingen (DE); Imo Hofer, March (DE)

Correspondence Address: STERNE, KESSLER, GOLDSTEIN & FOX PLLC, 1100 NEW YORK AVENUE, N.W., SUITE 600, WASHINGTON, DC, 20005-3934, US

(21) Appl. No.: 09/805,432

(22) Filed: Mar. 14, 2001

(51) Int. Cl. A61K 48/00; A61K 38/18; A61K 38/19; A61K 38/20

(52) U.S. Cl. 424/93.21; 514/2; 424/85.1; 424/85.2; 514/44

(57) ABSTRACT

The present invention relates generally to the modulation of arteriogenesis and/or the growth of collateral arteries or other arteries from preexisting arteriolar connections. In particular, the present invention provides a method for enhancing arteriogenesis and/or the growth of collateral arteries and/or other arteries from preexisting arteriolar connections comprising contacting an organ, tissue or cells with transforming growth factor beta 1 (TGFβ1) or a nucleic acid molecule encoding TGFβ1. The present invention also relates to the use of TGFβ1 or a nucleic acid molecule encoding TGFβ1 for the preparation of pharmaceutical compositions for enhancing arteriogenesis and/or collateral growth of collateral arteries and/or other arteries from preexisting arteriolar connections. Furthermore, the present invention relates to a method for the treatment of tumors comprising contacting an organ, tissue or cells with an agent which suppresses arteriogenesis and/or the growth of collateral arteries and/or other arteries from preexisting arteriolar connections through the inhibition of the biological activity of TGFβ1. The present invention further involves the use of an agent which suppresses arteriogenesis and/or the growth of collateral arteries and/or other arteries from preexisting arteriolar connections through the inhibition of the biological activity of TGFβ1 for the preparation of pharmaceutical compositions for the treatment of tumors.

The present invention relates generally to the modulation of arteriogenesis and/or the growth of collateral arteries or other arteries from preexisting arteriolar connections. In particular, the present invention provides a method for enhancing arteriogenesis and/or the growth of collateral arteries and/or other arteries from preexisting arteriolar connections comprising contacting an organ, tissue or cells with transforming growth factor beta 1 (TGFβ1) or a nucleic acid molecule encoding TGFβ1. The present invention also relates to the use of TGFβ1 or a nucleic acid molecule encoding TGFβ1 for the preparation of pharmaceutical compositions for enhancing arteriogenesis and/or collateral growth of collateral arteries and/or other arteries from preexisting arteriolar connections. Furthermore, the present invention relates to a method for the treatment of tumors comprising contacting an organ, tissue or cells with an agent which suppresses arteriogenesis and/or the growth of collateral arteries and/or other arteries from preexisting arteriolar connections through the inhibition of the biological activity of TGFβ1. The present invention further involves the use of an agent which suppresses arteriogenesis and/or the growth of collateral arteries and/or other arteries from preexisting arteriolar connections through the inhibition of the biological activity of TGFβ1 for the preparation of pharmaceutical compositions for the treatment of tumors.

Several documents are cited throughout the text of this specification. Each of the documents cited herein (including any manufacturer's specifications, instructions, etc.) are hereby incorporated herein by reference; however, there is no admission that any document cited is indeed prior art as to the present invention.

In the treatment of subjects with arterial occlusive diseases most of the current treatment strategies aim at ameliorating their effects. The only curative approaches involve angioplasty (balloon dilatation) or bypassing surgery. The former carries a high risk of restenosis and can only be performed in certain arterial occlusive diseases, like ischemic heart disease. The latter is invasive and also restricted to certain kinds of arterial occlusive diseases. There is no established treatment for the enhancement of arteriogenesis and/or collateral growth.

Vascular growth in adult organisms proceeds via two distinct mechanisms, sprouting of capillaries (angiogenesis) and in situ enlargement of preexisting arteriolar connections into true collateral arteries (Schaper, J. Collateral Circulation—Heart, Brain, Kidney, Limbs. Boston, Dordrecht, London: Kluwer Academic Publishers; 1993). Recent studies have disclosed mechanisms leading to angiogenesis with vascular endothelial growth factor (VEGF) as a major component (Tuder, J. Clin. Invest. 95 (1995), 1798-1807; Plate, Nature 359 (1992), 845-848; Ferrara, Endocrine Reviews 13 (1992), 1842; Klagsbrun, Annu. Rev. Physiol. 53 (1991), 217-239; Leung, Science 246 (1990), 1306-1309). This specific endothelial mitogen is upregulated by hypoxia and is able to promote vessel growth when infused into rabbit hindlimbs after femoral artery excision (Takeshita, J. Clin. Invest. 93 (1994), 662-670; Bauters, Am. J. Physiol. 267 (1994), H1263-H1271). These studies however did not distinguish between capillary sprouting, a mechanism called angiogenesis, and true collateral artery growth. Whereas VEGF is only mitogenic for endothelial cells, collateral artery growth requires the proliferation of endothelial and smooth muscle cells and pronounced remodeling processes occur (Schaper, J. Collateral Circulation—Heart, Brain, Kidney, Limbs. Boston, Dordrecht, London: Kluwer Academic Publishers; 1993; Jakeman, J. Clin. Invest. 89 (1992), 244-253; Peters, Proc. Natl. Acad. Sci. USA 90 (1993), 8915-8919; Millauer, Cell 72 (1993), 835-846; Pasyk, Am. J. Physiol. 242 (1982), H1031-H1037). Furthermore mainly capillary sprouting is observed in ischemic territories for example in the pig heart or in rapidly growing tumors (Schaper, J. Collateral Circulation—Heart, Brain, Kidney, Limbs. Boston, Dordrecht, London: Kluwer Academic Publishers; 1993; Plate, Nature 359 (1992), 845-848; Bates, Curr. Opin. Genet. Dev. 6 (1996), 12-19; Bates, Curr. Opin. Genet. Dev. 6 (1996), 12-19; Görge, Basic Res. Cardiol. 84 (1989), 524-535). True collateral artery growth, however, is temporally and spacially dissociated from ischemia in most models studied (Schaper, J. Collateral Circulation—Heart, Brain, Kidney, Limbs. Boston, Dordrecht, London: Kluwer Academic Publishers; 1993; Paskins-Hurlburt, Circ. Res. 70 (1992), 546-553). Other or additional mechanisms as those described for angiogenesis in ischemic territories are therefore needed to explain collateral artery growth. From previous studies it is known that these collateral arteries grow from preexisting arteriolar connections (Schaper, J. Collateral Circulation—Heart, Brain, Kidney, Limbs. Boston, Dordrecht, London: Kluwer Academic Publishers; 1993).

However, while agents such as VEGF and other growth factors are presently being employed to stimulate the development of angiogenesis after arterial occlusion, such agents are not envisaged as being capable of modulating the growth of preexisting arteriolar connections into true collateral arteries.

Thus, the technical problem of the present invention is to provide means and methods for the modulation of arteriogenesis and/or the growth of collateral arteries and/or other arteries from preexisting arteriolar connections.

The solution to this technical problem is achieved by providing the embodiments characterized in the claims.

Accordingly, the present invention is a method for enhancing arteriogenesis and/or growth of collateral arteries and/or other arteries from preexisting arteriolar connections comprising contacting organs, tissue or cells with transforming growth factor beta 1 (TGFβ1) and/or a nucleic acid molecule encoding said TGFβ1.

In the context of this invention the term “transforming growth factor beta 1” or “TGFβ1” refers to proteins and peptides which act on macrophages and which are capable of promoting collateral artery growth by direct activation, proliferation and/or potentiation of the effector functions of resident and newly recited macrophages on blood vessels. The present invention also comprises substances which are functionally equivalent to TGFβ1 in that these substances are capable of electing the aforementioned biological responses. The action of the TGFβ1 employed in the present invention may not be limited to the above-described specificity but they may also act on, for example eosinophils, lymphocyte subpopulations and/or stem cells.

In accordance with the present invention, a strong arteriogenic effect was found upon exogenous application of TGF-β₁ in vivo after femoral artery ligation. The number of collateral arteries on the x-ray angiograms as well as the conductance of the collateral vessels showed a significant increase upon TGF-β₁ treatment. In-vitro experiments showed activation and adhesion of monocytes which were accompanied by upregulation of the moncyte/macrophage adhesion receptor Mac-1 but no chemo-attractive activity of TGF-β₁, over a layer of endothelial cells.

The in-vivo arteriogenic effects of TGF-β₁ observed in accordance with the present invention are caused by activation of monocytes, leading to an increased adhesion, migration and subsequently perivascular accumulation of monocytes/macrophages. It has been found in accordance with the invention that said adhesion is inter alia due to increased expression of the adhesion receptor Mac-1. Adhesion and transmigration of monocytes/macrophages are initial steps in the process of arteriogenesis. In a further step production of various growth factors, such as basic-fibroblast growth factor (b-FGF), Platlet derived growth factor (PDGF), tumor necrosisi factor alpha (TNFa), Interleukine 1 (II-1), Interleukine 6 (IL-6) or vascular endothelial growth factor (VEGF) is stimulated in or by said monocytes/macrophages. Moreover, arteriogenesis is also effected by direct stimulation of vascular smooth muscle cells and/or endothelial cells by TGF-β1. Thus, in addition to the initiation of arteriogenesis due to the stimulation of the monocyte/macrophage pathway, arteriogenesis is further influenced by TGF-β1 due to the direct stimulation of the vascular smooth muscle cells and/or the endothelial cells in accordance with the present invention.

To the best of the inventor's knowledge, this is the first report disclosing TGF-β₁as a third specific arteriogenic substance, next to MCP-1 and the aforementioned CSFs, acting via the monocytic pathway, wherein TGF-β₁ increases arteriogenesis via activation of monocytes and induction of MAC-1 expression.

Advantageously, macrophages/monocytes can be efficiently activated by TGFβ1 and can subsequently adhere due to the upregulation of Mac-1 expression. Thereby, arteriogenesis via the macrophage/monocyte pathway is initiated and can be efficiently simulated in vivo.

The TGFβ1 to be employed in the methods and uses of the present invention may be obtained from various sources described in the prior art; see, e.g., Klagsbrun, Annu. Rev. Physiol. 53 (1991), 217-239. The potential exists, in the use of recombinant DNA technology, for the preparation of various derivatives of TGFβ1 comprising a functional part thereof or proteins which are functionally equivalent to TGFβ1. In this context, as used throughout this specification “functional equivalent or “functional part” of TGFβ1 means a protein having part or all of the primary structural conformation of TGFβ1 possessing at least the biological property of promoting at least one macrophage or granulocyte effector function mentioned above. The functional part of said protein or the functionally equivalent protein may be a derivative by way of amino acid deletion(s), substitution(s), insertion(s), addition(s) and/or replacement(s) of the amino acid sequence, for example by means of site directed mutagenesis of the underlying DNA. Recombinant DNA technology is well known to those skilled in the art and described, for example, in Sambrook et al. (Molecular cloning; A Laboratory Manual, Second Edition, Cold Spring Harbor Laboratory Press, Cold Spring Harbor N.Y. (1989)). Modified CSFs are described, e.g., in Yamasaki, Journal of Biochemistry 115 (1994), 814-819.

TGFβ1 or functional parts thereof or proteins which are functionally equivalent thereto, may be produced by known conventional chemical syntheses or recombinant techniques employing the amino acid and DNA sequences described in the prior art; see, e.g., EP-A-0 177 568; Han, Source Gene 175 (1996), 101-104; Kothari, Blood Cells, Molecules & Diseases 21 (1995), 192-200; Holloway. European Journal of Cancer 30A (1994), 2-6. For example, TGFβ1 may be produced by culturing a suitable cell or cell line which has been transformed with a DNA sequence encoding upon expression under the control of regulatory sequences TGFβ1 or a functional part thereof or a protein which is functionally equivalent TGFβ1. Suitable techniques for the production of recombinant proteins are described in. e.g., Sambrook, supra. Methods for constructing TGFβ1 and proteins as described above useful in the methods and uses of the present invention by chemical synthetic means are also known to those of skill in the art.

In another embodiment the invention relates to the use of transforming growth factor beta 1 (TGFβ1) and/or a nucleic acid molecule encoding said TGFβ1 for the preparation of a pharmaceutical composition for enhancing arteriogenesis and/or collateral growth of collateral arteries and/or other arteries from preexisting arteriolar connections.

The pharmaceutical composition comprises at least TGFβ1 as defined above, and optionally a pharmaceutically acceptable carrier or exipient. Examples of suitable pharmaceutical carriers are well known in the art and include phosphate buffered saline solutions, water, emulsions, such as oil/water emulsions, various types of wetting agents, sterile solutions etc. Compositions comprising such carriers can be formulated by conventional methods. The pharmaceutical compositions can be administered to the subject at a suitable dose. The dosage regimen may be determined by the attending physician considering the condition of the patient, the severity of the disease and other clinical factors. Administration of the suitable compositions may be effected by different ways, e.g. by intravenous, intraperetoneal, subcutaneous, intramuscular, topical or intradermal administration. The dosage regimen will be determined by the attending physician and other clinical factors. As is well known in the medical arts, dosages for any one patient depends upon many factors, including the patient's size, body surface area, age, the particular compound to be administered, sex, time and route of administration, general health. and other drugs being administered concurrently. Generally, the regimen as a regular administration of the pharmaceutical composition should be in the range of 1 μg to 10 mg units per day. If the regimen is a continuous infusion, it should also be in the range of 1 μg to 10 mg units per kilogram of body weight per minute, respectively. Progress can be monitored by periodic assessment. Dosages will vary but a preferred dosage for intravenous administration of DNA is from approximately 10⁶ to 10¹² copies of the DNA molecule. The compositions of the invention may be administered locally or systemically. Administration will generally be parenterally, e.g., intravenously; DNA may also be administered directly to the target site, e.g., by biolistic delivery to an internal or external target site or by catheter to a site in an artery.

In a preferred embodiment, TGFβ1 used in the methods and uses of the invention is a recombinant TGFβ1. DNA sequences for TGFβ1 which can be applied in the methods and uses of the invention are known in the prior art and described in e.g. Ohta, Biochem. J. 350 (2000), 395-404. Moreover, DNA and amino acid sequences of TGFβ1 are available in the Gene Bank database. As described above, methods for the production of recombinant proteins are well-known to the person skilled in the art; see, e.g., Sambrook, supra.

In a further preferred embodiment, the method and the use of the present invention is designed to be applied in conjugation with a growth factor or cytokine comprising fibroblast growth factor (FGF), preferably b-FGF, platelet derived growth factor (PDGF), tumor necrosis Factor alpha (TNFα), interleukin 1 (IL-1), Interleukin 6 (IL-6), or vascular endothelial growth factor (VEGF). This embodiment is particularly suited for enhancing of both sprouting of capillaries (angiogenesis) and in situ enlargement of preexisting arteriolar connections into true collateral arteries. Pharmaceutical compositions comprising TGFβ1, and a growth factor such as VEGF may be used for the treatment of peripheral vascular diseases or coronary artery disease.

The nucleic acid and amino acid sequences of said growth factors or cytokines are well known in the art and are available e.g. in the GeneBank database.

In another preferred embodiment, the method of the invention comprises

(a) obtaining cells, tissue or an organ from a subject;

(b) introducing into said cells, tissue or organ a nucleic acid molecule encoding and capable of expressing the TGFβ1 in vivo; and

(c) reintroducing the cells, tissue or organ obtained in step (b) into the same subject or a different subject.

It is envisaged by the present invention that the TGFβ1 and the nucleic acid molecules encoding said proteins are administered either alone or in combination, and optionally together with a pharmaceutically acceptable carrier or exipient. Said nucleic acid molecules may be stably integrated into the genome of the cell or may be maintained in a form extrachromosomally, see, e.g., Calos, Trends Genet. 12 (1996), 463-466. On the other hand, viral vectors described in the prior art may be used for transfecting certain cells, tissues or organs.

Furthermore, it is possible to use a pharmaceutical composition of the invention which comprises a nucleic acid molecule encoding TGFβ1 in gene therapy. Suitable gene delivery systems may include liposomes, receptor-mediated delivery systems, naked DNA, and viral vectors such as herpes viruses, retroviruses, adenoviruses, and adeno-associated viruses, among others. Delivery of nucleic acid molecules to a specific site in the body for gene therapy may also be accomplished using a biolistic delivery system, such as that described by Williams (Proc. Natl. Acad. Sci. USA 88 (1991), 2726-2729).

Standard methods for transfecting cells with nucleic acid molecules are well known to those skilled in the art of molecular biology, see, e.g., WO 94/29469. Gene therapy to prevent or decrease the development of diseases described herein may be carried out by directly administering the nucleic acid molecule encoding TGFβ1 to a patient or by transfecting cells with said nucleic acid molecule ex vivo and infusing the transfected cells into the patient. Furthermore, research pertaining to gene transfer into cells of the germ line is one of the fastest growing fields in reproductive biology. Gene therapy, which is based on introducing therapeutic genes into cells by ex-vivo or in-vivo techniques is one of the most important applications of gene transfer. Suitable vectors and methods for in-vitro or in-vivo gene therapy are described in the literature and are known to the person skilled in the art; see, e.g., Giordano, Nature Medicine 2 (1996), 534-539; Schaper, Circ. Res. 79 (1996), 911-919; Anderson, Science 256 (1992), 808-813; Isner, Lancet 348 (1996). 370-374; Muhlhauser, Circ. Res. 77 (1995), 1077-1086; Wang, Nature Medicine 2 (1996), 714-716; WO94/29469; WO 97/00957 or Schaper, Current Opinion in Biotechnology 7 (1996), 635-640, and references cited therein. The nucleic acid molecules comprised in the pharmaceutical composition of the invention may be designed for direct introduction or for introduction via liposomes, or viral vectors (e.g. adenoviral, retroviral) containing said nucleic acid molecule into the cell. Preferably, said cell is a germ line cell, embryonic cell, or egg cell or derived therefrom.

It is to be understood that the introduced nucleic acid molecules encoding the TGFβ1 express said proteins after introduction into said cell and preferably remain in this status during the lifetime of said cell. For example, cell lines which stably express said TGFβ1 may be engineered according to methods well known to those skilled in the art. Rather than using expression vectors which contain viral origins of replication, host cells can be transformed with the recombinant DNA molecule or vector of the invention and a selectable marker, either on the same or separate vectors. Following the introduction of foreign DNA, engineered cells may be allowed to grow for 1-2 days in an enriched media, and then are switched to a selective media. The selectable marker in the recombinant plasmid confers resistance to the selection and allows for the selection of cells having stably integrated the plasmid into their chromosomes and grow to form foci which in turn can be cloned and expanded into cell lines. This method may advantageously be used to engineer cell lines which express TGFβ1. Such cells may be also be administered in accordance with the pharmaceutical compositions, methods and uses of the invention.

A number of selection systems may be used, including but not limited to the herpes simplex virus thymidine kinase (Wigler, Cell 11(1977), 223), hypoxanthine-guanine phosphoribosyltransferase (Szybalska, Proc. Natl. Acad. Sci. USA 48 (1962), 2026), and adenine phosphoribosyltransferase (Lowy, Cell 22 (1980), 817) in tk⁻. hgprt⁻ or aprt⁻ cells, respectively. Also, antimetabolite resistance can be used as the basis of selection for dhfr, which confers resistance to methotrexate (Wigler, Proc. Natl. Acad. Sci. USA 77 (1980), 3567; O'Hare, Proc. Natl. Acad. Sci. USA 78 (1981), 1527), gpt, which confers resistance to mycophenolic acid (Mulligan, Proc. Natl. Acad. Sci. USA 78 (1981), 2072); neo, which confers resistance to the aminoglycoside G-418 (Colberre-Garapin, J. Mol. Biol. 150 (1981), 1); hygro, which confers resistance to hygromycin (Santerre, Gene 30 (1984), 147); or puromycin (pat, puromycin N-acetyl transferase). Additional selectable genes have been described, for example, trpB, which allows cells to utilize indole in place of tryptophan; hisD, which allows cells to utilize histinol in place of histidine (Hartman, Proc. Natl. Acad. Sci. USA 85 (1988), 8047); and ODC (ornithine decarboxylase) which confers resistance to the ornithine decarboxylase inhibitor, 2-(difluoromethyl)-DL-ornithine, DFMO (McConlogue, 1987, In: Current Communications in Molecular Biology, Cold Spring Harbor Laboratory ed.).

Thus, in a preferred embodiment, the nucleic acid molecule comprised in the pharmaceutical composition for the use of the invention is designed for the expression of TGFβ1 by cells in vivo by, for example, direct introduction of said nucleic acid molecule or introduction of a plasmid, a plasmid in liposomes, or a viral vector (e.g. adenoviral, retroviral) containing said nucleic acid molecule.

In a preferred embodiment of the method and uses of the present invention, the TGFβ1 derivative or functional equivalent substance is an antibody, (poly)peptide, nucleic acid, small organic compound, ligand, hormone, PNA or peptidomimetic.

In this context, it is understood that TGFβ1 to be employed according to the present invention may be, e.g., modified by conventional methods known in the art. For example, it is possible to use fragments which retain the biological activity of TGFβ1 as described above, namely the capability of promoting collateral artery growth. This further allows the construction of chimeric proteins and peptides wherein other functional amino acid sequences may be either physically linked by, e.g., chemical means to TGFβ1 or may be fused by recombinant DNA techniques well known in the art. Furthermore, folding simulations and computer redesign of structural motifs of the TGFβ1 as well as their respective receptors can be performed using appropriate computer programs (Olszewski, Proteins 25 (1996), 286-299; Hoffman, Comput. Appl. Biosci. 11 (1995), 675-679). Computer modeling of protein folding can be used for the conformational and energetic analysis of detailed receptor and protein models (Monge, J. Mol. Biol. 247 (1995), 995-1012; Renouf, Adv. Exp. Med. Biol. 376 (1995), 37-45). In particular, the appropriate programs can be used for the identification of interactive sites of TGFβ1 and their respective receptors by computer assistant searches for complementary peptide sequences (Fassina, Immunomethods 5 (1994), 114-120). Further appropriate computer systems for the design of protein and peptides are described in the prior art, for example in Berry, Biochem. Soc. Trans. 22 (1994), 1033-1036; Wodak, Ann. N.Y. Acad. Sci. 501 (1987), 1-13; Pabo, Biochemistry 25 (1986), 5987-5991. The results obtained from the above-described computer analysis can be used for, e.g., the preparation of peptidomimetics of TGFβ1, or fragments thereof. Such pseudopeptide analogues of the natural amino acid sequence of the protein may very efficiently mimic the parent protein or peptide (Benkirane, J. Biol. Chem. 271 (1996), 33218-33224). For example, incorporation of easily available achiral Ω-amino acid residues into TGFβ1 protein or a fragment thereof results in the substitution of amide bonds by polymethylene units of an aliphatic chain, thereby providing a convenient strategy for constructing a peptidomimetic (Banerjee, Biopolymers 39 (1996), 769-777). Superactive peptidomimetic analogues of small peptide hormones in other systems are described in the prior art (Zhang, Biochem. Biophys. Res. Commun. 224 (1996), 327-331). Appropriate peptidomimetics may also be identified by the synthesis of peptidomimetic combinatorial libraries through successive amide alkylation and testing the resulting compounds, e.g., according to the methods described in the prior art. Methods for the generation and use of peptidomimetic combinatorial libraries are described in the prior art, for example in Ostresh. Methods in Enzymology 267 (1996), 220-234 and Dorner, Bioorg. Med. Chem. 4 (1996), 709-715. Furthermore, antibodies or fragments thereof may be employed which, e.g., upon binding to a TGFβ1-receptor mimic the biological activity of the receptor's ligand.

Furthermore, a three-dimensional and/or crystallographic structure of the TGFβ1 or of its receptors can be used for the design of peptidomimetic inhibitors of the biological activity of a CSF (Rose, Biochemistry 35 (1996), 12933-12944; Rutenber, Bioorg. Med. Chem. 4 (1996),1545-1558).

In a preferred embodiment, the methods and uses of the invention may be employed for diseases caused by a vascular disease or a cardiac infarct or a stroke or for any disease where an increase of blood supply via collaterals, arteries etc. is needed.

In a particularly preferred embodiment, the methods and uses of the invention are designed to be applied to a subject suffering from arteriosclerosis, a coronary artery disease, a cerebral occlusive disease, a peripheral occlusive disease, a visceral occlusive disease, renal occlusive disease, a mesenterial arterial insufficiency or an ophthamic or retenal occlusion or for any disease where atherosclerotic plaques in the vascular wall lead to an obstruction of the vessel diameter.

In a further preferred embodiment, the methods and uses of the invention are designed to be applied to a subject during or after exposure to an agent or radiation or surgical treatment which damage or destroy arteries.

As discussed above, arteriogenesis and the growth of arteries from preexisting arteriolar connections is essential for the delivery of nutrition to tumors. Thus, if the growth of said vessels to the tumor would be suppressed suppression and/or inhibition of tumor growth is to be expected.

Accordingly, the invention relates to a method for the treatment of tumors comprising contacting organs, tissue or cells with an agent which suppresses arteriogenesis and/or the growth of collateral arteries and/or other arteries from preexisting arteriolar connections though inhibition of the biological activity of TGFβ1 as defined above.

The explanations and definitions of the terms herein above apply mutatis mutandis to the aforementioned method and the following method and use claims.

As discussed above, macrophages play a pivotal role during arteriogenesis and/or the growth of collateral arteries and/or other arteries from preexisting arteriolar connections. TGFβ1 stimulates macrophages/monocytes and increases adhesion of said cells to the enothelial cells of the blood vessels inter alia via increased expression of the adhesion receptor Mac-1. Transmigration and adhesion to the endothelial cells are the initial steps of arteriogenesis as occurs during tumor formation. In a further step, growth factors and cytokines comprising those referred to herein above are released due to said stimulation of the macrophages/monocytes by TGFβ1. As is evident from the above, by inhibition of TGFβ1 arteriogenesis can be efficiently suppressed at the initial steps and tumor formation and progression is inhibited.

Advantageously, by identifying TGFβ1 as a trigger molecule in accordance with the present invention it is now possible to treat tumor diseases caused or influenced by arteriogenesis and/or the growth of collateral arteries and/or other arteries from preexisting arteriolar connections by the methods and uses referred to herein above and below.

Moreover, the invention relates to the use of an agent which suppresses the growth of collateral arteries and/or other arteries from preexisting arteriolar connections through the inhibition of the biological activity of TGFβ1 as defined above for the preparation of a pharmaceutical composition for the treatment of tumors.

In a more preferred embodiment of the method or use of the invention the agent inhibits the biological activity of TGFβ1 and/or inhibits an intracellular signal or signal cascade comprising SMAD proteins triggered in macrophages through the receptor for TGFβ1.

The term “SMAD” proteins used in accordance with the present invention refers to a family of signal transducers and transcription factors which are activated intracellularly by the TGFβ receptors upon stimulation by TGFβ1. These signal transducers are either directly or indirectly involved in the activation of TGFβ1 target genes and hence in elicting a biological response. Thus, the expression of growth factors or Mac1 protein may be stimulated and/or induced by said SMAD proteins.

An agent which inhibits the biological activity of TGFβ1 also inhibits the intracellular transducing of the signal by SMAD proteins upon binding of TGFβ1 to its receptor on target cells.

SMAD proteins are well known in the art. Nucleic acid or amino acid sequences are available, e.g., in the database GeneBank. Moreover, it has been reported that blood vessels are a pivotal expression site of said SMAD proteins in the developing mouse embryo (Dick, Developmental Dynamics, 211 (1998), 293-305).

In a more preferred embodiment of the use of the invention, the agent blocks interaction of TGFβ1 and its receptor.

Receptors for TGFβ1 are well known in the art and have been described in, e.g.. Lin et al., Mol. Reprod. Dev. 32 (1992), 105-110; Nilsen-Hamilton et al., New Biol. 4 (1992), 127-131. Moreover, amino acid and nucleic acid sequences are provided by the Gene Bank database.

In a preferred embodiment, the agent used in the methods and uses of the invention is a(n) antibody, (poly)peptide, nucleic acid, small organic compound, ligand, hormone, PNA or peptidomimetic.

Nucleic acid molecules specifically hybridizing to TGFβ1 encoding genes and/or their regulatory sequences may be used for repression of expression of said gene, for example due to an antisense or triple helix effect or they may be used for the construction of appropriate ribozymes (see, e.g., EP-B1 0 291 533, EP-A1 0 321 201, EP-A2 0 360 257) which specifically cleave the (pre)-mRNA of a gene encoding a CSF. The nucleic and amino acid sequences encoding TGFβ1 are known in the art and described, for example, in Han, Source Gene 175 (1996), 101-104; Kothari, Blood Cells, Molecules & Diseases 21 (1995), 192-200 or in Holloway, European Journal of Cancer 30A (1994), 2-6. Selection of appropriate target sites and corresponding ribozymes can be done as described for example in Steinecke, Ribozymes, Methods in Cell Biology 50, Galbraith et al. eds Academic Press, Inc. (1995), 449-460.

Nucleic acids comprise DNA or RNA or hybrids thereof. Furthermore, said nucleic acid may contain, for example, thioester bonds and/or nucleotide analogues, commonly used in oligonucleotide anti-sense approaches. Said modifications may be useful for the stabilization of the nucleic acid molecule against endo- and/or exonucleases in the cell. Furthermore, the so-called “peptide nucleic acid” (PNA) technique can be used for the inhibition of the expression of a gene encoding a TGFβ1. For example, the binding of PNAs to complementary as well as various single stranded RNA and DNA nucleic acid molecules can be systematically investigated using, e.g., thermal denaturation and BlAcore surface-interaction techniques (Jensen, Biochemistry 36 (1997), 5072-5077). The synthesis of PNAs can be performed according to methods known in the art, for example, as described in Koch, J. Pept. Res. 49 (1997), 80-88; Finn, Nucleic Acids Research 24 (1996), 3357-3363. Furthermore, folding simulations and computer redesign of structural motifs of TGFβ1 and its receptor can be performed as described above to design drugs capable of inhibiting the biological activity of TGFβ1.

Furthermore, antibodies may be employed specifically recognizing TGFβ1 or its receptor or parts, i.e. specific fragments or epitopes, of TGFβ1 and its receptor thereby inactivating the TGFβ1 or its receptor. These antibodies can be monoclonal antibodies, polyclonal antibodies or synthetic antibodies as well as fragments of antibodies, such as Fab, Fv or scFv fragments etc. Antibodies or fragments thereof can be obtained by using methods which are described, e.g., in Harlow and Lane “Antibodies, A Laboratory Manual”, CSH Press, Cold Spring Harbor, 1988 or EP-B1 0 451 216 and references cited therein. For example, surface plasmon resonance as employed in the BlAcore system can be used to increase the efficiency of phage antibodies which bind to an epitope of TGFβ1 or its receptor (Schier, Human Antibodies Hybridomas 7 (1996), 97-105; Malmborg, J. Immunol. Methods 183 (1995), 7-13).

Putative inhibitors which can be used in accordance with the present invention including peptides, proteins, nucleic acids, antibodies, small organic compounds, ligands, hormones, peptidomimetics, PNAs and the like capable of inhibiting the biological activity of TGFβ1 or its receptor may be identified according to the methods known in the art, for example as described in EP-A-0 403 506 or in the appended examples.

In a preferred embodiment, methods and uses of the invention are employed for the treatment of a tumor which is a vascular tumor, preferably selected from the group consisting of Colon Carcinoma, Sarcoma, Carcinoma in the breast, Carcinoma in the head/neck, Mesothelioma, Glioblastoma, Lymphoma and Meningeoma.

In a preferred embodiment, the pharmaceutical composition in the use of the invention is designed for administration by catheter intraarterial, intravenous, intraperitoneal or subcutenous routes. In the examples of the present invention the TGFβ1 protein was for instance administered locally via osmotic minipump.

These and other embodiments are disclosed or are obvious from and encompassed by the description and examples of the present invention. Further literature concerning any one of the methods, uses and compounds to be employed in accordance with the present invention may be retrieved from public libraries, using for example electronic devices. For example the public database “Medline” may be utilized which is available on Internet, e.g. under http://www.ncbi.nlm.nih.gov/PubMed/medline.html. Further databases and addresses, such as http://www.ncbi.nim.nih.gov/, http://www.infobiogen.fr/, http://www.fmi.ch/biology/research_tools.html, http://lwww.tigr.org/, are known to the person skilled in the art and can also be obtained using, e.g., http://www.lycos.com. An overview of patent information in biotechnology and a survey of relevant sources of patent information useful for retrospective searching and for current awareness is given in Berks, TIBTECH 12 (1994), 352-364.

The use and methods of the invention can be used for the treatment of all kinds of diseases hitherto unknown as being related to or dependent on the modulation of arteriogenesis and/or the growth of collateral arteries and/or other arteries from preexisting arteriolar connections. The methods and uses of the present invention may be desirably employed in humans, although animal treatment is also encompassed by the methods and uses described herein. Moreover, the methods and uses encompassed by the present invention may be applied in vivo or in vitro.

The figures show

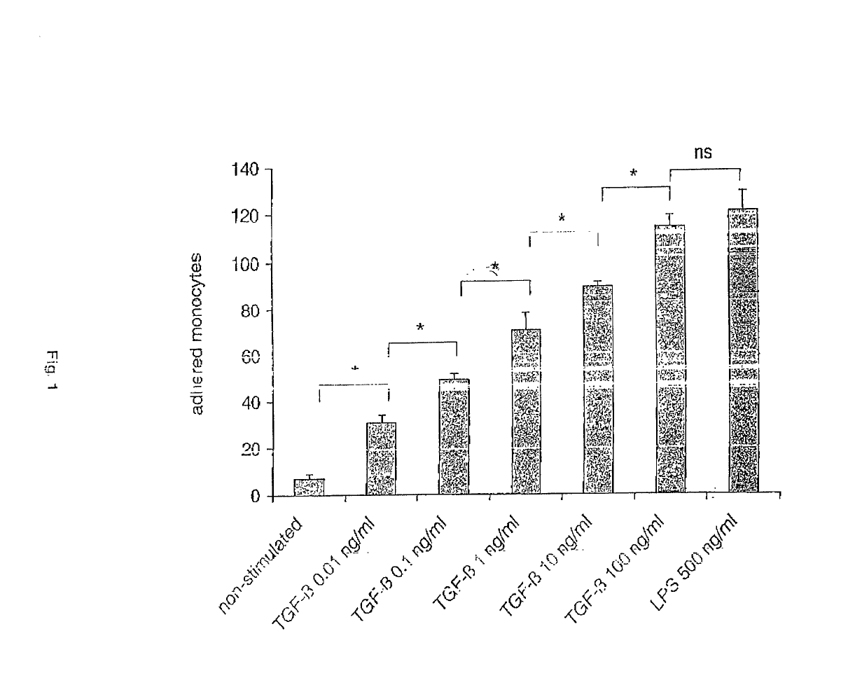

FIG. 1: Stimulation of monocytes with TGF-β₁ leads to a dose-dependent increase in adhesion to a monolayer of endothelial cells.

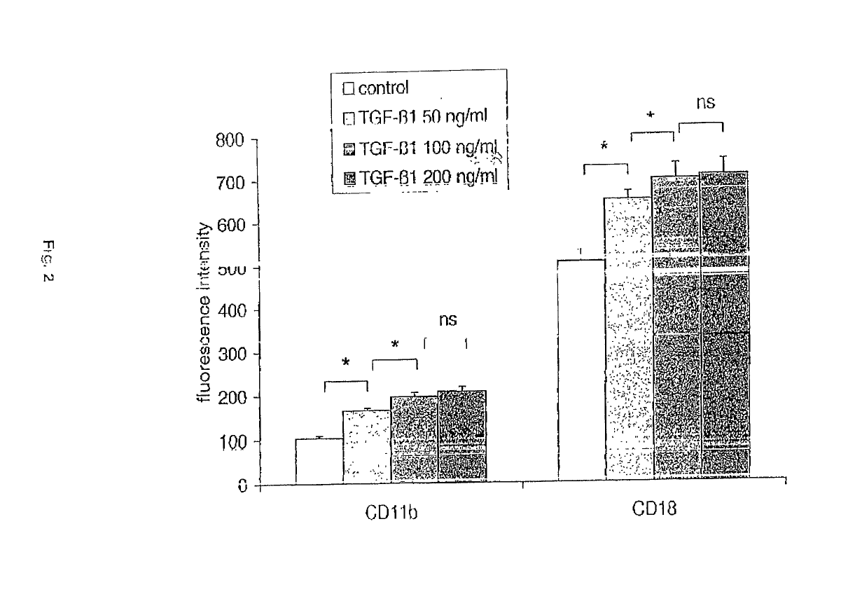

FIG. 2: MAC-1 receptor expression on monocytes significantly increases upon TGF-β₁ stimulation.

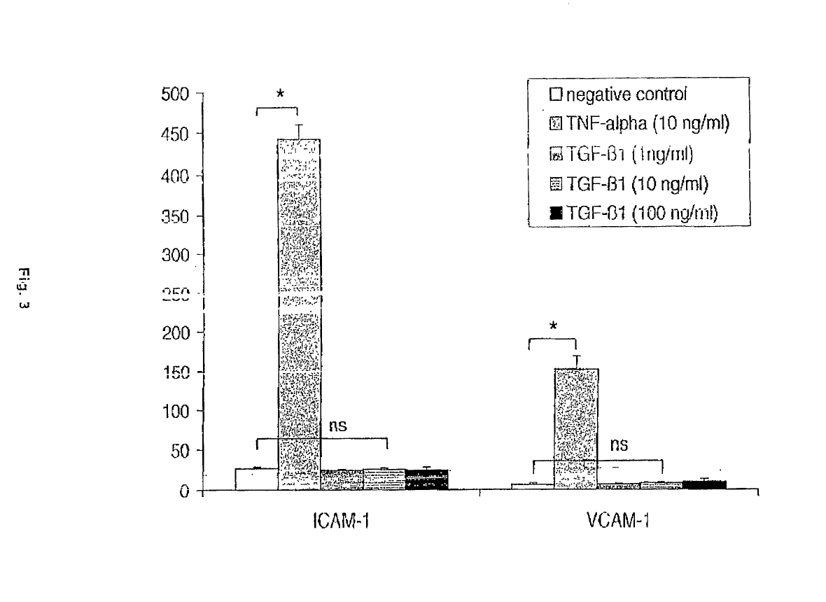

FIG. 3: TGF-β treatment of EC's causes no increase in ICAM or VCAM expression

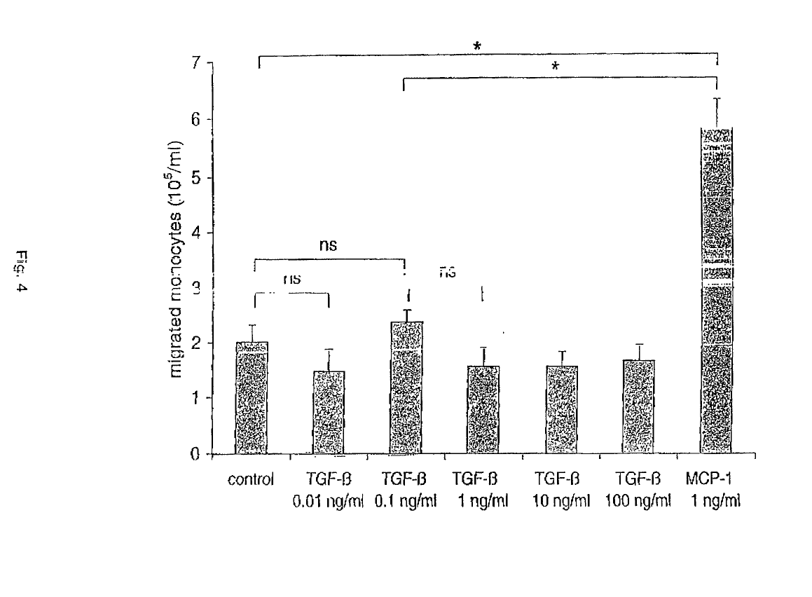

FIG. 4: TGF-β₁ exerts no chemo-attractivity towards monocytes over a layer of smooth muscle cells.

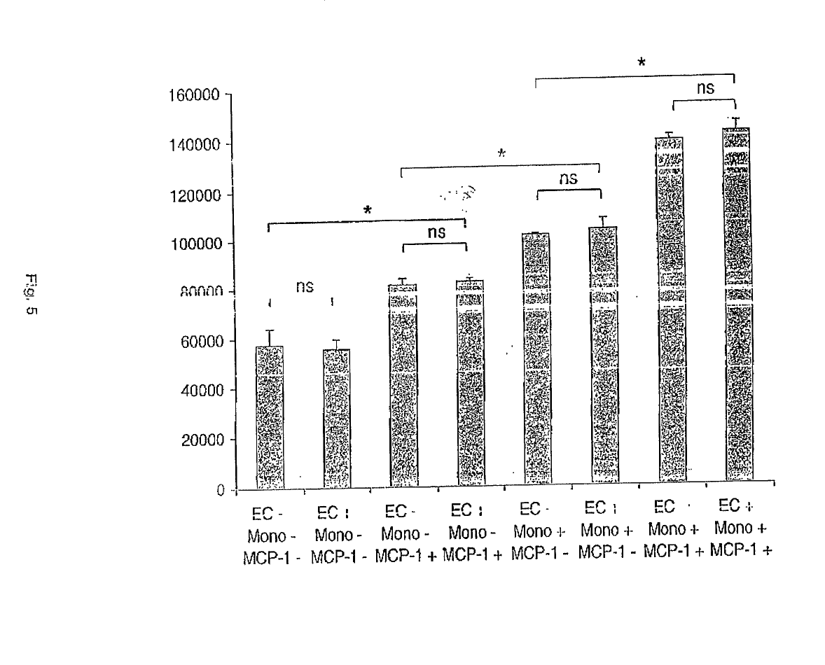

FIG. 5: Maximum migration of monocytes over a layer of endothelial cells is achieved after pre-stimulation with TGF-β₁ and using MCP-1 for the chemo-attractive gradient.

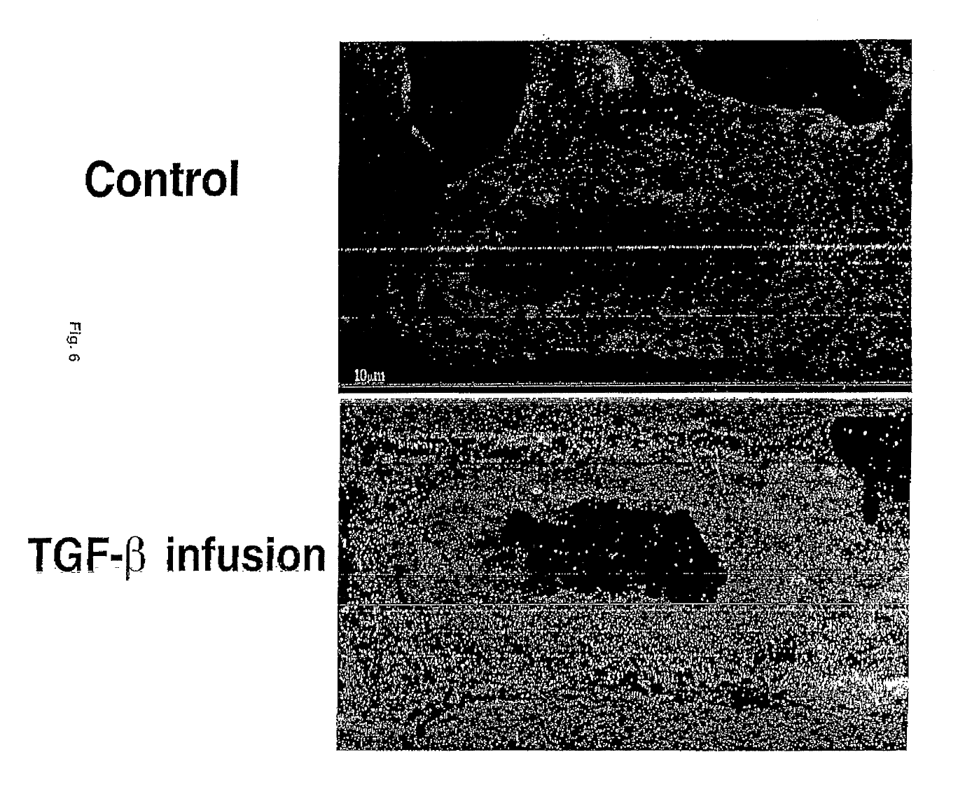

FIG. 6: 6a shows an infiltrating cell around a growing collateral artery in a control animal, expressing TGF-β₁. In the treated animal, TGF-β₁ is abundantly present around the growing collateral artery (6b).

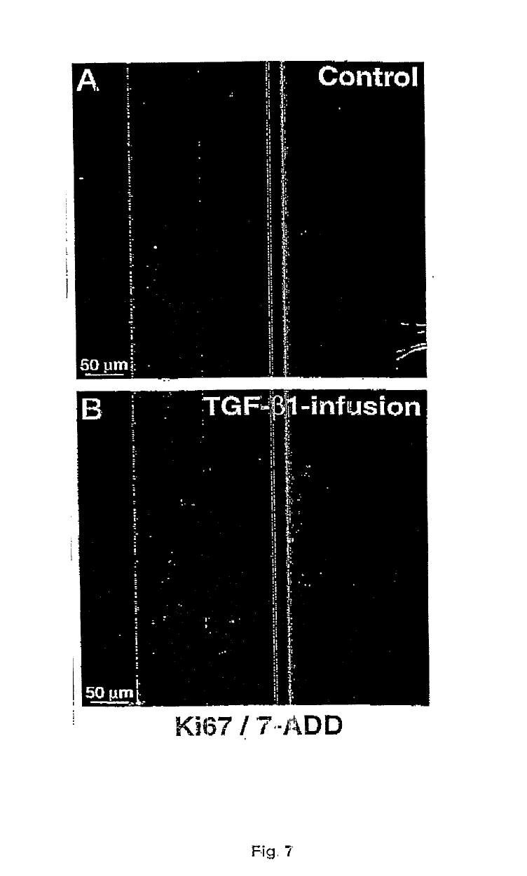

FIG. 7: Immunolabeling for Ki-67 (green) in growing collateral arteries in control and TGF-β₁ treated animals. Nuclei are labelled red with 7-AAD. Notice higher levels of immunodetectable Ki-67 positive cells within or around growing collateral arteries in TGF-β₁ treated rabbits as compared with control group.

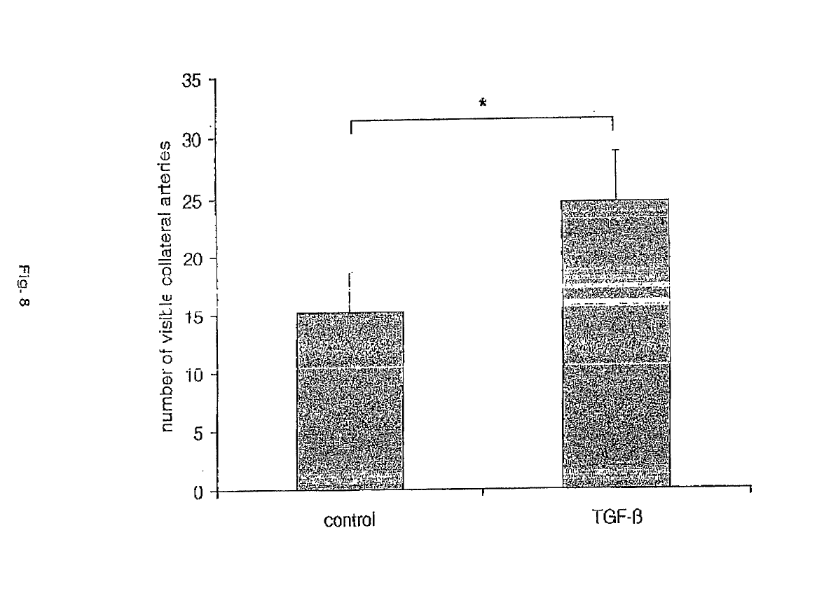

FIG. 8: Total number of visible collateral arteries is increased upon TGF-β₁ treatment when quantified under stereoscopic viewing.

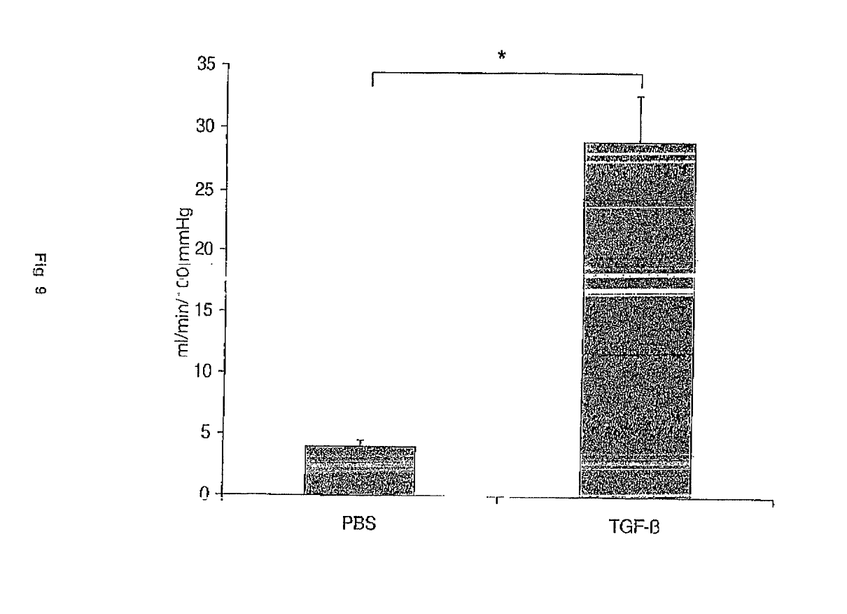

FIG. 9: Collateral conductance,one week after ligation of the femoral artery in the rabbit, increases about sevenfold upon TGF-β₁ treatment.

The examples illustrate the invention.

### EXAMPLE 1

### Adhesion and Transmigration Assays

Monocytes were isolated from buffy coats of healthy blood donors by density gradient centrifugation and elutriation as described previously (Heil et al., Eur J. Cell Biol. 2000). Human umbilical endothelial cells (HUVECs) were prepared according to the method of Jaffe et al.(J. Clin. Invest. 52 (1973), 2745-2756) and were cultivated as described elsewhere (Heil et al., loc. cit.).

Adhesion assays were performed as previously described (Heil et al., loc. cit.). Monocytes were stimulated for two hours with TGF-β₁ (concentrations; 0.01, 0.1, 1, 10 and 100 ng/ml) (PeproTech, London, UK) or LPS (positive control). To identify the effects of TGF-β₁ stimulation of endothelial cells on the adhesion of monocytes, HUVECs were either stimulated with TNF-α (positive control, 10 ng/ml, Sigma, Deisenhofen, FRG) or with different doses of TGF-β₁.

Transmigration assays were performed as previously described (Heil et al., loc. cit.) to test the chemoattractive potency of TGF-β₁ over a layer of endothelial cells. In a second set of transmigration assays the influence of monocyte-stimulation and/or endothelium-stimulation with TGF-β₁ was determined.

A strongly increased adhesion to the HUVEC layer was observed after stimulation of monocytes with TGF-β₁. The adhesion of monocytes was linearly related to TGF-β₁ dose (FIG. 1). The maximally achieved adhesion of monocytes upon TGF-β₁ treatment was similar to the adhesion observed for the positive control (LPS: 120.2±8.3 cells/field vs. TGF-β₁: 114.0±4.7 p=NS). The treatment of the HUVECs layer with TGF-β₁ caused no increase in the number of adhered monocytes as compared to the control. This was confirmed by FACS analysis, showing no significant increase in the expression of either ICAM, VCAM or P-selectin on endothelial cells treated with TGF-β₁ (FIG. 3).

TGF-β₁ showed no chemoattractive potency towards monocytes in the trans-endothelial migration assays. When TGF-β₁ was diluted at different concentrations into the lower chamber of the assay, the migration of monocytes did not differ significantly from the control assay and was significantly lower as compared to MCP-1 (FIG. 4). Also when the HUVEC-layer was stimulated with TGF-β₁ no increase in the number of transmigrated cells was observed. However, when monocytes were pre-stimulated with TGF-β₁ an increased trans-endothelial migration of monocytes was observed as compared to the control group. When monocytes and endothelium were stimulated simultaneously with TGF-β₁ the transmigration rate was similar to that after monocyte stimulation alone. Maximum migration of monocytes was achieved when MCP-1 was added to the lower chamber of the transmigration assay, in combination with TGF-β₁ stimulation of monocytes (FIG. 5).

### EXAMPLE 2

### Expression of Adhesion Molecules on Monocytes and Endothelial Cells

The expression of the MAC-1 receptor significantly increased, dose-dependently, after stimulation of human monocytes with TGF-β₁ (FIG. 2). A similar dose-dependent response of MAC-1 expression (CD11b/CD18) upon TGF-β₁ stimulation was found in rabbit monocytes (control: 91.2±4.2/482±21.7; TGF-β₁ 50 ng/ml: 129,3±3,8/553,3±17,9; TGF-β₁ 100 ng/ml: 155.5±7.2/602.2±23.4; TGF-β₁ 200 ng/ml 193.9±6.7/675.5±25.7, p<0.05 for all differences).

### EXAMPLE 3

### In-Vivo Arteriogenesis

36 New Zealand White Rabbits (NZWR) were randomly assigned to one of three groups (n=12 each). In two groups the femoral artery was ligated and either Phosphate Buffered Saline (PBS) or TGF-β₁ (0.48 μg/kg/d) (PeproTech, London, UK) was delivered locally, directly into the collateral circulation, via an osmotic minipump as previously described (Hoefer et. al., accepted for publication, Cardiovascular Research, 2001). To obtain the normal conductance value and angiographic appearance of the vascular tree of the rabbit hindlimb, the third group was evaluated without ligation. For final experiments animals of each group were randomly assigned to either angiographic or hemodynamic measurements. X-ray angiograms were performed as previously described (Longland, Ann. Roy. Coll. Surg. Engl. 13 (1953), 161-164). Following Longland's definition, only vessels showing a defined stem, midzone and re-entry, identifying them as collateral arteries, were counted (Longland, loc. cit.). Hemodynamic measurements and calculations of collateral conductance were performed as previously described (Hoefer et. al., accepted for publication, Cardiovascular Research, 2001) using fluorescent microspheres and FACS-analysis.

An additional six animals were operated as described above and treated with either PBS (n=3) or TGF-β₁ (n=3). Three days after ligation of the femoral artery animals were sacrificed and tissue was harvested from the hindlimb muscles for histological examination. For the rate of proliferation, sections were stained with Ki-67 (Monotec, mouse derived). Alpha-smooth muscle actin was detected using FITC-conjugated alpha-SM antibody (clone 1A4, Sigma). For the detection of TGF-β₁ around the growing collateral arteries a mouse-derived TGF-β₁ antibody was used (clone MAB 240, R&D systems). TOTO-3 and 7-aminoactinomycin D (Molecular Probes) were used for nuclear staining. Tissue samples were examined by CSLM using Leica TCSNT, equipped with argon/krypton and helium/neon lasers.

Significant differences between sample means were determined with a two-tailed Student's T-test. Differences with a p-value<0.05 were classified as significant.

No animals were lost during or after femoral artery ligation. Gangrene or gross impairment of hindlimb function after femoral artery occlusion was also not observed. The body weights and body temperature within the different groups did not show any significant difference. There were no detectable differences in the values of total protein, albumin, glutamic oxaloacetic transaminase and glutamic pyruvic transaminase.

Three days after ligation of the femoral artery, increased levels of TGF-beta-1 were noted within and around growing collateral arteries (FIG. 6A) whereas in tissue sections of the non-occluded hindlimb, TGF-β₁ could rarely be detected (data not shown). The level of immunodetectable TGF-β₁ was conspicuously increased within and around growing collateral arteries in TGF-β₁ treated animals (FIG. 6B). Immunolabeling for Ki-67 revealed higher numbers of proliferating cells in growing collateral arteries after TGF-β₁ infusion as compared with the non-treated control group (FIG. 7).

Angiograms performed one week after ligation of the femoral artery showed several, typically corkscrewed, collateral arteries spanning from the arteria profunda femoris and the arteria circumflexa femoris to the arteria genualis and the arteria saphena parva. TGF-β₁ infusion for a time-period of one week had significantly increased the number of visible collateral arteries as compared to the PBS-control group (FIG. 8; total number of visible collateral arteries: PBS; 15.2±3.4, TGF-β₁; 24.6±4.1, p<0.05). One week after femoral artery ligation collateral conductance in the control group was 4.1±0.5 ml/min/100 mmHg. TGF-β₁ had significantly increased collateral conductance to over 6-fold as compared to the PBS-treated group (25.6±3.7 ml/min/100 mmHg, FIG. 9). In the non-occluded control group a conductance value of 161.5±10.8 ml/min/100 mmHg was measured.

The results of the experiments performed in accordance with the present invention indicate that TGFβ1 is capable of mediating arteriogenesis and/or the growth of collateral arteries and/or other arteries from preexisting arteriolar connections by activation of the moncyte/macrophage pathway. This activation is accompanied by expression of the adhesion receptor Mac-1. Due to the experiments referred to above, the present invention provides novel means and methods for the treatment of disease by modulation of arteriogenesis and/or the growth of collateral arteries and/or other arteries from preexisting arteriolar connections.

The present invention is not to be limited in scope by its specific embodiments described which are intended as single illustrations of individual aspects of the invention and any proteins, nucleic acid molecules, or compounds which are functionally equivalent are within the scope of the invention. Indeed, various modifications of the invention in addition to those shown and described therein will become apparent to those skilled in the art from the foregoing description and accompanying drawings. Said modifications intended to fall within the scope of the appended claims. Accordingly, having thus described in detail preferred embodiments of the present invention, it is to be understood that the invention defined by the appended claims is not to be limited to particular details set forth in the above description as many apparent variations thereof are possible without departing from the spirit or scope of the present invention.

1. A method for enhancing arteriogenesis and/or the growth of collateral arteries and/or other arteries from preexisting arteriolar connections comprising contacting organs, tissue or cells with transforming growth factor beta 1 (TGFβ1) and/or a nucleic acid molecule encoding said TGFβ1.

2. Use of transforming growth factor beta 1 (TGFβ1) and/or a nucleic acid molecule encoding said TGFβ1 for the preparation of a pharmaceutical composition for enhancing arteriogenesis and/or collateral growth of collateral arteries and/or other arteries from preexisting arteriolar connections.

3. The method of claim 1 or the use of claim 2, wherein the TGFβ1 is a recombinant TGFβ1.

4. The method of claims 1 or 3, further comprising contacting the organ, tissue or cell with a growth factor or cytokine.

5. The use of claims 2 or 3, wherein the pharmaceutical composition is designed to be administered in conjugation with a growth factor or cytokine.

6. The method of claim 4 or the use of claim 5, wherein said growth factor or cytokine is b-FGF, PDGF, TNF-α, IL-1, IL-6 or VEGF.

7. The method of any one of claims 1, 3, 4 or 6, comprising
(a) obtaining cells, tissue or an organ from a subject;
(b) introducing into said cells, tissue or organ a nucleic acid molecule encoding and capable of expressing TGFβ1 in vivo; and
(c) reintroducing the cells, tissue or organ obtained in step (b) into the same subject or a different subject.

8. The method of any one of claims 1, 3, 4, 6 or 7 or the use of any one of claims 2, 3, 5 or 6, wherein the TGFβ1 is a derivative or functional equivalent substance.

9. The method or use of claim 8, wherein said derivative or functional equivalent substance is an antibody, (poly)peptide, nucleic acid, small organic compound, ligand, hormone, PNA or peptidomimetic.

10. The method of any one of claims 1, 3, 4, 6 to 9 or the use of any one of claims 2, 3, 5, 6, 8 or 9, wherein said method or said pharmaceutical composition is designed to be applied to a subject suffering from a vascular disease or a cardiac infarct or a stroke.

11. The method or the use of claim 10, wherein said vascular disease is arteriosclerosis and/or a hyperlipidemic condition, a coronary artery disease, cerebral occlusive disease, peripheral occlusive disease, visceral occlusive disease, renal artery disease, mesenterial arterial insufficiency or an ophtamic or retenal occlusion.

12. The method of any one of claims 1, 3, 4, 6 to 11 or the use of any one of claims 2, 3, 5, 6, 8 to 11, wherein said method or said pharmaceutical composition is designed to be applied to a subject during or after exposure to an agent or radiation or surgical treatment which damage or destroy arteries.

13. A method for the treatment of tumors comprising contacting organs, tissue or cells with an agent which suppresses arteriogenesis and/or the growth of collateral arteries and/or other arteries from preexisting arteriolar connections through inhibition of the biological activity of TGFβ1 as defined in any one of claims 1 to 12.

14. Use of an agent which suppresses the growth of collateral arteries and/or other arteries from preexisting arteriolar connections through the inhibition of the biological activity of TGFβ1 as defined in any one of claims 1 to 12 for the preparation of a pharmaceutical composition for the treatment of tumors.

15. The method of claim 13 or the use of claim 14, wherein the agent inhibits the biological activity of TGFβ1 and/or inhibits an intracellular signal or signal cascade comprising SMAD proteins triggered in macrophages through the receptor for TGFβ1.

16. The method or the use of claim 15, wherein the agent blocks an interaction of the TGFβ1 and its receptor.

17. The method of any one of claims 13, 15 or 16 or the use of any one of claims 14 to 16, wherein the agent is derived from a class of substances as defined in claim 9.

18. The method or the use of claim 17, wherein the agent is designed to be expressed in vascular cells or cells surrounding preexisting arteriolar connections to a tumor.

19. The method of any one of claims 13 or 15 to 18 or the use of any one of claims 14 to 18, wherein the tumor is a vascular tumor.

20. The method or the use claim 19, wherein the tumor is selected form the group consisting of Colon Carcinoma, Sarcoma, Carcinoma in the breast, Carcinoma in the head/neck, Mesothelioma, Glioblastoma, Lymphoma and Meningeoma.

21. The use of any one of claims 2, 3, 5, 6, 8 to 11, 14 to 20, wherein the pharmaceutical composition is designed to be administered by intracoronary, intramuscular, intraarterial, intravenous, intraperitoneal or subcutenous routes.
